(19) United States
(12) Patent Application Publication
(10) Pub. No.: US 2022/0268465 A1
(43) Pub. Date: Aug. 25, 2022

(54) SANITIZING CLOSED AIR ENVIRONMENT WITH OZONE-TREATED STEAM FROM WATER DISTILLATION APPARATUS WITH OZONE GENERATOR

(71) Applicant: John C. ELLIS, JR., Westbrookville, NY (US)

(72) Inventor: John C. ELLIS, JR., Westbrookville, NY (US)

(21) Appl. No.: 17/677,263

(22) Filed: Feb. 22, 2022

Related U.S. Application Data
(60) Provisional application No. 63/152,469, filed on Feb. 23, 2021.

(51) Int. Cl. F24F 8/26; F24F 13/30

(52) U.S. Cl. CPC F24F 8/26; F24F 13/30

(57) ABSTRACT

Provided is a method for sanitizing a closed air environment by using a portion of vapor (or steam) produced in a water distillation apparatus which includes an ozone generator while reducing ozone emission into the closed air environment.

### CROSS-REFERENCE TO RELATED APPLICATIONS

This application claims priority to U.S. Provisional Application No. 63/152,469, filed Feb. 23, 2021.

### FIELD

This disclosure relates to use of a portion of vapor (or steam) produced in a water distillation apparatus which includes an ozone generator for sanitizing a closed air environment while reducing ozone emission into the closed air environment.

### SUMMARY

One embodiments is a method for sanitizing a closed air environment, the method comprises: providing a distillation apparatus comprising a water reservoir container having a water inlet and outlet thereto connected so as to create a reservoir of water therein and a boiler smaller than the reservoir container with an open connection between the container and the boiler for water flow to or from the boiler, a steam line overhead of the water level in the boiler leading to a condenser immersed in the reservoir, an outlet from said condenser to discharge condensate and an ozone generator in said boiler; maintaining a predetermined level of reservoir water by introducing feed water into and removing water from said container at a flow rate which maintains a temperature in the range of about 180°-190° F. in the reservoir and heating the water in said boiler to generate steam, the steam passing through ozone generated by said ozone generator and passing into said condenser to be condensed therein by heat exchange against the reservoir water whereby the reservoir water becomes heated and the heated reservoir water becomes devolatilized and deaerated prior to entering said boiler; and releasing a portion of the steam that passed through ozone generated by said ozone generator into the closed air environment to sanitize the closed air environment. In some embodiments, the distilling apparatus may be inside of the closed air environment. Yet in some embodiments, the distillation apparatus may be positioned apart from the closed air environment and the portion of steam passes from the distillation apparatus through a conduit to the closed air environment. In such a case, the portion of the steam that passed through ozone may be released into the closed air environment using, for example, a pipe, which provides a fluidic connection between the apparatus and the closed air environment. Such pipe may be, for example, connected to an outlet of a valve releasing the portion of the steam that passed through ozone.

### FIGURES

FIG. 1 is a fragmentary plan view of one embodiment of the distilling apparatus;

FIG. 2 is a cross sectional view of FIG. 1 taken along the line 2-2 thereof;

FIG. 3 is a cross sectional view of FIG. 1 taken along the line 3-3 thereof and illustrates air circulating means carried by the container; and

FIG. 4 is a cross sectional view showing the boiler and a fragmentary portion of the tank taken along the line 4-4 of FIG. 2.

FIG. 5 is a diagrammatic view showing the condenser tube containing a deflector.

FIG. 6 is a diagrammatic view showing the fluid conduit containing a deflector.

FIG. 7 is a photograph of a modified degasification and distillation apparatus, which includes fluidically connected container 10 and boiler 12. The boiler 12 of the modified apparatus includes a poppet valve 90 on the top. The black knob of valve 90 may be used for adjusting a value of released pressure. When open, valve 90 may fluidically connect the inner volume of boiler 12 and the closed air environment directly (when the apparatus is inside the closed air environment) or using an additional pipe.

### DETAILED DESCRIPTION

Ozone may be used for sanitizing a closed air environment, such as an environment, which does not have an effective air flow exchange with an outside environment. Examples of closed air environments include an inner space of a room or an inner space of a vehicle, such as a car. The effective air flow exchange for the closed air environment may be prevented by wall(s), closed door(s) and/or closed window(s). Exposure to ozone, however, may be harmful to living beings, such as humans and/or animals.

The present inventor suggests use of a portion of vapor (or steam) produced in a water distillation apparatus which includes an ozone generator for sanitizing a closed air environment. Examples of such distillation apparatus may be an apparatus for water degasification and distillation disclosed in U.S. Pat. No. 6,409,888, which is incorporated herein by reference; and a water treatment apparatus disclosed in U.S. Patent Application Publication No. 2015-0246825, which is incorporated herein by reference in its entirety. For sanitizing a closed air environment, a boiler of the distillation apparatus may be equipped with a pressure valve, which may open during a boiler pressure cycle, when an ozone treated vapor accumulates in the boiler, so that at least a portion of the ozone treated vapor is released into the closed air environment. The pressure valve, which may be positioned on the boiler, may close during a vacuum cycle during which water leaves the boiler. Opening and closing of the pressure valve may be repeated multiple, e.g. 2, 3, 4, 5 or more, times, for example, as water goes between the boiler and the condenser of the distillation apparatus. A time of an individual opening may be, for example, from 5 second to 10 minutes or from 10 seconds to 5 minutes or from 15 seconds to 3 minutes or any value or subrange within these ranges. The repetition rate of closing and opening the valve may be varied by varying one or more parameters of the apparatus, such as a power (wattage) of the heater, a size of the boiler's feeder tube, a size of the condensing coil and a diameter of the boiler. Using the ozone treated vapor instead of ozone for sanitizing may reduce harmful exposure to ozone.

As used herein, sanitizing a closed environment may mean killing microorganisms, such as viruses and/or bacteria.

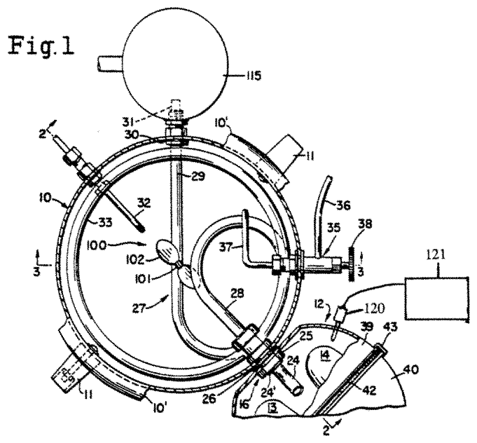

An exemplary water distillation apparatus which may be used for sanitizing a closed air environment is illustrated in FIGS. 1-7.

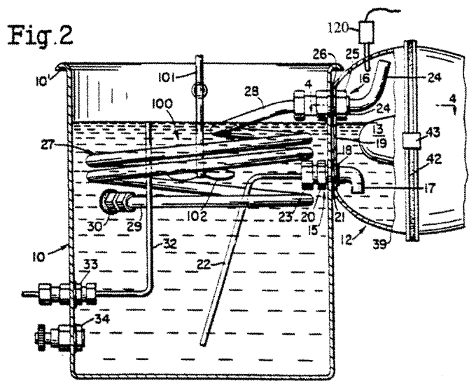

Referring now to the drawings and more specifically to FIGS. 1 and 2 it may be seen that the distilling apparatus comprises a cylindrical tank reservoir container 10 having a pair of handles 11 secured to the side thereof. A boiler 12 having instantaneous heating elements 13 and 14 therein is affixed to the side of the tank 10 by the fluid connectors 15 and 16. The fluid connector 15 includes an elbow 17 having a shoulder 18 and a threaded shank extending through cooperating openings in the wall 19 of the boiler 12 and the wall of the container 10. A nut 20 engages the shank of the fitting 15 and together with a resilient washer 21 provides a water-tight seal for both the tank 10 and the boiler 12. A water inlet conduit 22 is fixedly coupled to the fitting 15 by means of a nut 23 so that water within reservoir container 10 will automatically feed into the boiler 12 until the water level within boiler 12 corresponds to the reservoir water level inside reservoir container 10. It will also be observed that the water level is maintained at a level adequate to effect total or at least substantial immersion of the heating elements 13 and 14 in the water of boiler 12.

The steam outlet fitting 16 is of conventional construction and includes an outlet pipe 24, a threaded shank 24′ extending through the walls of reservoir container 10 and boiler 12 and secured thereto by a nut 25.

A sealing washer 26 is disposed between container and boiler to provide a watertight connection. In the preferred embodiment, the condenser 27 is in the form of a coiled tube of metal such as stainless steel, copper or the like and has the inlet end portion 28 sealably connected to the fitting 16 within container 10. The outlet 29 of the condenser 27 has a fitting 30 extending through the wall of container 10 and provides the condensate outlet 31. Optionally, but desirably an oversized filter 115 is interposed at condensate outlet 31. As best may be seen in FIG. 1, the coiled condenser tubing generates a cylindrical region 100 at the center of the reservoir container 10. Reservoir container 10 further includes an overflow pipe 32 which is connected to a fitting 33 sealed to the wall of the container 10 and a drain cock 34 for draining water from the container (being useful for cleaning and maintenance). A water inlet valve 35 is at the upper portion of the reservoir container 10 and has an inlet 36, an outlet 37 and a hand-wheel 38 for regulating the water supply in order to maintain an appropriate supply of water to the reservoir container 10.

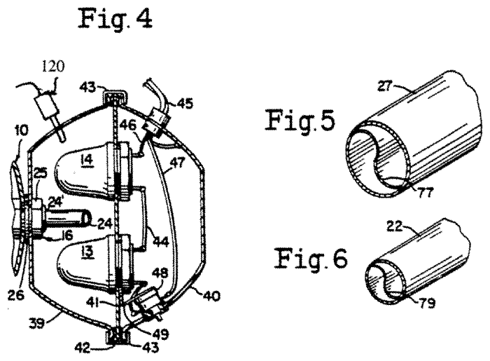

The boiler 12 is shown more clearly in FIG. 4 and in the preferred embodiment illustrated herein includes two housing elements 39 and 40. A wall 41 disposed between the housing elements 39 and 40 includes a peripheral seal 42 which is releasably clamped between the outer rims of the housing elements 39 and 40 by clips 43 at the periphery of the boiler 12. This arrangement completely seals the boiler formed by the housing element 39 and the wall 41. The whole boiler can be readily disassembled for cleaning. The heating elements 13 and 14 in the illustrated embodiment are carried by the wall 41 and are connected in series by a lead 44 connecting one terminal of one heater to one terminal of the other heater. The power line 45 has one lead 46 connected to the other terminal of the heater 14 while the second lead 47 is connected through a thermostat 48 to the other terminal of the heater 13. The thermostat is mounted on a bracket 49 in close proximity to the heaters 13. In the event the heater 13 reaches a temperature above the normal operating temperature, the thermostat will operate to open the circuit and de-energize both heaters 13 and 14. It is evident, however, that the heaters 13 and 14 could be arranged for parallel operating or in the alternative a single electric heater may be employed in the boiler provided however it delivers the quantity of heat necessary for operation of the distillation apparatus.

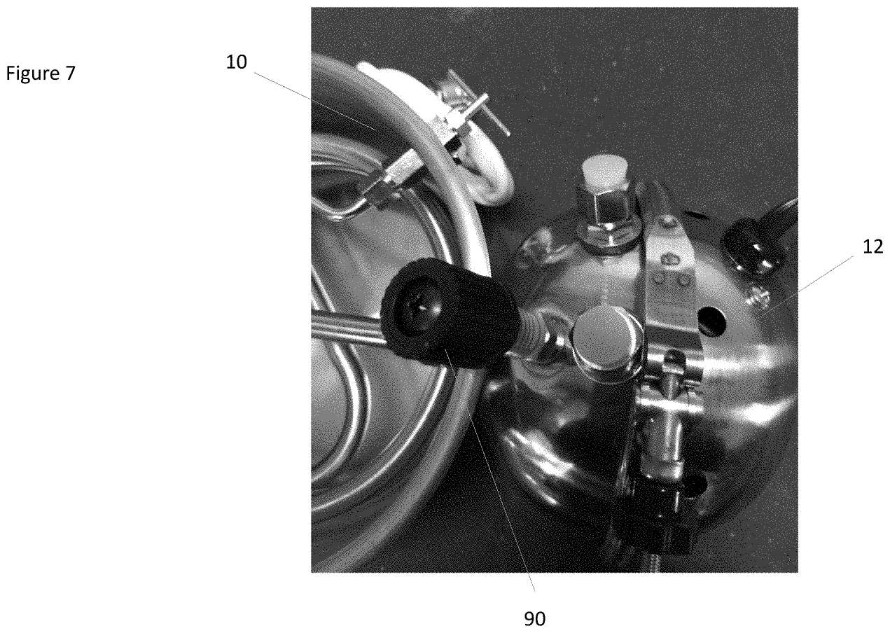

The boiler 12 includes a pressure release valve 90 for releasing a portion of ozone treated steam for sanitizing a closed air environment, see FIG. 7. The pressure release valve 90 may be, for example, a spring loaded pressure valve, such as a poppet valve. A particular pressure at which valve 90 releases the ozone treated steam may be adjusted by, for example, adjusting a knob setting of the poppet valve.

Included in the boiler is the ozone generator 120. Preferably, the ozone generator is inserted into the boiler through a port in housing element 39. As illustrated in FIG. 1, the ozone generator 120 is powered by transformer 121. It is not necessary, however, to have a separate power supply for the ozone generator. The ozone generator may be powered by the same power supply used to operate heaters 13 and 14.

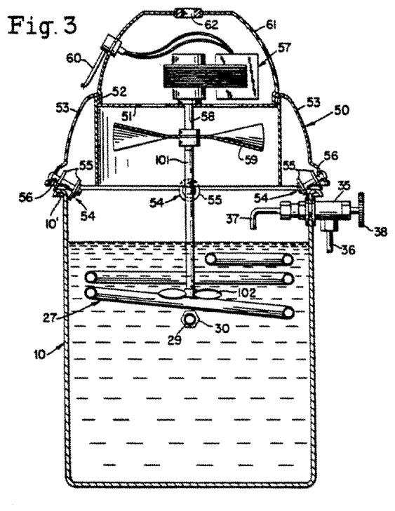

In the preferred embodiment, a forced air circulation means assists removal of steam and undesirable vapors liberated from the reservoir water within reservoir container 10. The air circulating means which is shown in FIG. 3 comprises an inverted dished cover generally denoted by the numeral 50 over reservoir container 10 which includes a flat upper wall 51 that is perforated or apertured, an upwardly extending peripheral wall 52 and a downwardly curved peripheral wall 53. The lower peripheral edge of the wall 53 carries three or more diagonally disposed rollers 54 each having spaced discs 55 rotatably carried by a shaft 56. The discs 55 engage the rolled edge 10′ of the reservoir container 10 and accordingly provide an annular vent between the cover 50 and the top edge of the reservoir container 10.

The flat apertured wall 51 of the air circulating means supports an electric motor generally denoted by the numeral 57 which powers a shaft 58 extending through the perforated wall 51. The fan 59 is mounted on shaft 58. Power is fed to the motor 57 by a cable 60 connected in a conventional manner to the motor. If desired, switch means may be provided for operation of the fan. The fan motor 57 is covered by a vented dome-shaped housing 61 that is securely fitted to the cover 50 and is attached thereto by any suitable means. In the illustrated embodiment, the dome-shaped housing 61 frictionally engages the peripheral wall 52 of the cover 50.

In one mode of fan operation, air is drawn into the air circulating means assembly through an opening 62 in the dome-shaped housing 61 and then down through the perforated wall 51 whereupon it is directed downwardly over the reservoir water in reservoir container 10 and thereafter is discharged through the annular opening between the reservoir container 10 and its cover 50. In the reverse mode of fan operation the fan 59 draws air in through the annular opening between reservoir container 10 and its cover 50 up through perforated wall 51 and opening out through vent 62 in motor housing 61.

As may be seen in FIG. 3, a stirrer rod 101 extends from an integral connection with motor shaft 58 at the hub of fan 59 preferably but not necessarily axially of the cylindrical reservoir region 100 inside of condenser coil 27 and terminates at the stirrer blades 102 immersed in the reservoir water. The depth of immersion for stirrer blades 102 is not critical, but preferably, they are not deeper than the bottom of coil condenser 27. In the mode illustrated herein, the stirrer rod was positioned modestly off-center to avoid interference with the outlet bend 29 of condenser coil 27, see FIGS. 1 and 2.

In the operation of the distillation apparatus, the reservoir container 10 and boiler 12 are first filled with water to a level at least substantially covering the heating elements 13 and 14 as may be observed most clearly in FIG. 2. It will be observed that when filling reservoir container 10, water will automatically flow through conduit 22 into the boiler so that ultimately the level of the water in the reservoir container 10 will be the same as the water level in the boiler 12. When energy is then supplied to the heating elements 13 and 14 they will function to boil the water within the boiler 12, which in some embodiments, may include one or more UV bulbs. Oxygen in the air above the water is turned to ozone by ozone generator 120. Steam generated from heating elements 13 and 14 rises through the ozone and enters inlet 24. A portion of the steam is then released via pressure release valve 90 to sanitize a closed environment in which the apparatus is positioned. The unleased portion of steam then flows through the condenser coil 27 to be condensed therein. The condensed steam will then discharge through filter 115 as the distillate (liquid) product from the condenser outlet 31. When first operating the distillation apparatus, it is generally desirable to discard the distillate product until the water inside reservoir container 10 has attained a normal operating temperature which preferably is 180°-190° F. (which is rapidly attained).

Heaters 13 and 14 are designed to heat the water within the boiler at a rate faster than the condenser coil 27 can accommodate the steam produced. Accordingly, a head of steam is developed within the boiler 12 and the steam pressure will force at least a portion of liquid from the boiler back through the conduit 22 into the reservoir container 10 thereby relieving the steam pressure. The flow of the water from the boiler into conduit 22 generates a vacuum in the boiler. The vacuum causes the closure of the pressure release valve 90. The vacuum also causes air to be drawn through filter 115 into the condenser outlet 31, traveling through the condenser and exiting in the boiler via outlet 24 and thereby providing fresh oxygen for the ozone generator 120. As soon as the steam pressure within the boiler is relieved, water will again flow through the conduit 22 back into the boiler with the result that there will be a periodic reversal of water flow through the conduit 22 and air flow through the condenser 27. This pulsating action results in a more rapid increase in temperature of the reservoir water within the container 10 by contributing heat over and above the heat imparted to the reservoir water by the action of the condenser coil 27. It also results in a constant renewing of ozone in the boiler. The temperature of the reservoir water, however, is always below the boiling temperature (of the water in boiler 12) so that distillate will be condensed in condenser 27. Preferably the reservoir water should be kept in the range of 180° F. to 190° F. This temperature level will boil off undesirable components from the reservoir water (prior to actual distillation thereof), and also serves to operate condenser 27 adequately. To maintain proper operation of the apparatus, a substantial proportion of the feed water which enters at the inlet 37 ultimately is discharged as overflow through tube 32 and outlet 35.

As has already been pointed out, a mechanical expedient to facilitate maintenance of a distinct temperature across the condenser coil tubing is illustrated in FIG. 5. Shown there is an enlarged partial cross-section of condenser coil tubing. Inside the tubing is a deflector 77 whose purpose is to generate spiral flow movement of steam and condensate to the tube wall. Also, flow becomes more turbulent thereby helping heat exchange across the tube wall. A like deflector 79 may be provided in the conduit connecting reservoir container 10 and boiler 12 (see FIG. 6). The purpose of deflector 77 is, of course, to create turbulent mixing of the water so as to avoid any temperature stratification either in reservoir container 10 or in boiler 12.

An additional optional expedient is the provision of a filter, preferably an oversized filter, at the condenser outlet 31. In the embodiment illustrated herein the oversized filter 115 is a carbon filter.

Filter 115 absorbs any organic materials that are carried over with the condensate. It polishes the condensate, so to speak but also it achieves a superior aeration for the condensate. As has already been pointed out the distillation apparatus operates in a pulsating fashion causing water to flow through the connecting conduit 22 back and forth between boiler 12 and reservoir 12. The same pulsations affect condenser 27. A pulse of (steam) pressure (“pressure cycle”) from boiler 12 passes through the condenser tubing in a forward direction during a steam generation pulse, sending condensate out through filter 115. Then during the reverse suction pulse (“vacuum cycle”), air is drawn into the filter 115, through the condenser, into the boiler. Thus, the filter 115 acts as much to filter air drawn into the condenser tubing, as it does to filter distillate leaving the condenser tubing. Opening and closing of the pressure valve releasing a portion of the ozone-treated steam from the boiler may be timed to correspond respectively to the pressure cycle or pulse and vacuum/suction cycle or pulse.

The pulses may be not equal in their effect. Steam is being generated in boiler 12, with a portion of steam released through valve 90 and a portion of steam being condensed in condenser coil 27. The distillate is discharged at the outlet 31 through filter 115. A net movement outflow movement of distilled water through filter 115 results. At the same time, a small net inflow of air into filter 115 and condenser 27 results. The distillate, e.g., at 190°-195° F., is hot enough to heat filter 115 and prevents microbial contamination of the filter. This means that air which enters oversized filter 115 during the suction pulses is retained therein and becomes sterilized by the hot filter before entering condenser 27 and/or becoming absorbed in the distillate. The reason for providing a filter 115 that may be oversized may be precisely to increase the residence time therein of the inflowing air. Overall, the result is that air heated and sterilized in filter 115 partially aerates the distilled water improving the palatability thereof. While only certain embodiments have been illustrated and described herein, it is understood that alterations, changes, and modifications may be made therein without departing from the true scope and spirit thereof.

1. A method for sanitizing a closed air environment, the method comprises:
providing a distillation apparatus comprising a water reservoir container having a water inlet and outlet thereto connected so as to create a reservoir of water therein and a boiler smaller than the reservoir container with an open connection between the container and the boiler for water flow to or from the boiler, a steam line overhead of the water level in the boiler leading to a condenser immersed in the reservoir, an outlet from said condenser to discharge condensate and an ozone generator in said boiler;
maintaining a predetermined level of reservoir water by introducing feed water into and removing water from said container at a flow rate which maintains a temperature in the range of about 180°-190° F. in the reservoir and heating the water in said boiler to generate steam, the steam passing through ozone generated by said ozone generator and passing into said condenser to be condensed therein by heat exchange against the reservoir water whereby the reservoir water becomes heated and the heated reservoir water becomes devolatilized and deaerated prior to entering said boiler; and
releasing a portion of the steam that passed through ozone generated by said ozone generator into the closed air environment to sanitize the closed air environment.

2. The method of claim 1, wherein the boiler of the distillation apparatus comprises a pressure valve and said releasing is performed though opening of the pressure valve.

3. The method of claim 2 wherein the boiler periodically generates more steam than can be accommodated by the condenser thereby causing a periodic pulsation wherein water is forced to flow out of the boiler back into the reservoir by steam pressure, and the steam pressure pulse is dissipated whereupon water flows back into the boiler from the reservoir, said pulsation includes the steam pressure pulse of a flow of condensate out of the condenser and a suction pulse of a flow of air into said boiler and wherein said opening of the pressure valve is timed with said steam pressure pulse and closing of the pressure valve is timed with the suction pulse.
